(19) United States
(12) Patent Application Publication
(10) Pub. No.: US 2022/0287517 A1
(43) Pub. Date: Sep. 15, 2022

(54) FABRIC WARMING CONTAINER

(71) Applicants: Joseph Schwartz, Hollywood, FL (US); Gunter Felix, Hialeah, FL (US)

(72) Inventors: Joseph Schwartz, Hollywood, FL (US); Gunter Felix, Hialeah, FL (US)

(21) Appl. No.: 17/198,547

(22) Filed: Mar. 11, 2021

(51) Int. Cl. A47K 10/06; A47K 10/10; H05B 3/26; A61L 2/10

(52) U.S. Cl. CPC A47K 10/06; A47K 10/10; H05B 3/26; A61L 2/10; A47K 2201/00; A61L 2202/26; A61L 2202/14

(57) ABSTRACT

A fabric warming container includes a base portion and one or more sidewall portions coupled to and extending vertically from the base portion along at least a portion of the periphery of the base portion. The one or more sidewall portions include an interior surface. One or more ultraviolet (UV) light sources may be positioned on the interior surface of the one or more sidewall portions.

### TECHNICAL FIELD

The present disclosure generally relates to fabric warming containers and, more particularly, for fabric warming containers with ultraviolet (UV) lights.

### BACKGROUND

Warm and sterile towels are not only desirable to have after bathing or showering, but may be required in a medical environment. It is well known that a warmed towel serves to offset the chilling effect due to evaporation of water from the body after showering, even at normal room temperatures.

With the rise of antibiotic resistant bacteria and other pathogens, cleanliness is of the utmost importance. Sterilizing a towel or fabric is the best way to ensure that there are no pathogens on a surface that is going to be in direct bodily contact. If a towel contaminated with a dangerous virus or bacteria came in contact with several individuals, the potential for the spread of disease is greatly increased. The soothing feeling that a warm towel provides after a bath or shower is positive reinforcement to a user to continue using warmed towels on a regular basis. By combining a sterilizing feature, a user will benefit from a clean sterile towel free of dangerous bacteria and other pathogens. Unfortunately, at this time there are no devices that both warm and disinfect towels that are adaptable to home or commercial use.

It may be beneficial to warm and sanitize towels or other fabrics without utilizing a bulky appliance or other large enclosure.

### SUMMARY

In an embodiment, a fabric warming container includes a base portion and one or more sidewall portions coupled to and extending vertically from the base portion along at least a portion of the periphery of the base portion. The one or more sidewall portions may include an interior surface. One or more ultraviolet (UV) light sources may be positioned on the interior surface of the one or more sidewall portions. The one or more sidewall portions may form an opening opposite to the base portion above a cavity defined by the one or more sidewall portions and the base portion.

One or more of the following features may be included. One or more heating elements may be positioned on the interior surface of the one or more sidewall portions. One or more reflector portions may be positioned on the interior surface of the one or more sidewall portions. The cavity defined by the one or more sidewall portions and the base portion may be configured to receive one or more fabrics. The one or more sidewall portions may include a single, continuous sidewall portion extending completely around the periphery of the base portion. The one or more sidewall portions may include a plurality of sidewall portions spaced apart from one another along at least a portion of the periphery of the base portion. The one or more sidewall portions may include one or more gaps in the one or more sidewall portions extending from the opening along the sidewall portion to the base portion. A timer component may be configured to control the one or more UV light sources. One or more shelves may be positioned within the cavity defined by the one or more sidewall portions. The fabric warming container may be configured to be coupled to an article of furniture, built into a wall, and/or mounted onto a wall.

According to another embodiment, a fabric warming container includes a base portion and a pair of sidewall portions coupled to and extending vertically from the base portion on opposing sides of the base portion. Each sidewall portion may include an interior surface. One or more ultraviolet (UV) light sources may be positioned on the interior surface of each sidewall portion. The pair of sidewall portions may form a pair of openings positioned on opposing sides of the base portion.

One or more of the following features may be included. One or more heating elements may be positioned on the interior surface of at least one of the sidewall portions. One or more reflector portions may be positioned on the interior surface of at least one of the sidewall portions. A timer component may be configured to control the one or more UV light sources. One or more shelves may be positioned between the pair of sidewall portions. The fabric warming container may be configured to be coupled to an article of furniture, built into a wall, and/or mounted onto a wall.

According to yet another embodiment, fabric warming container includes a base portion and one or more sidewall portions coupled to and extending vertically from the base portion along at least a portion of the periphery of the base portion. The one or more sidewall portions may include an interior surface. One or more ultraviolet (UV) light sources may be positioned on the interior surface of the one or more sidewall portions. The one or more sidewall portions and the base portion may define a cavity within the fabric warming container. The fabric warming container may be configured to be coupled to an article of furniture, built into a wall, and/or mounted onto a wall.

One or more of the following features may be included. One or more heating elements may be positioned on the interior surface of the one or more sidewall portions. One or more shelves may be positioned within the cavity defined by the one or more sidewall portions. One or more reflector portions may be positioned on the interior surface of at least one of the sidewall portions.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a fabric warming container, according to various example embodiments;

FIG. 2 is a diagrammatic view of the fabric warming container of FIG. 1 with broken lines to show the interior elements of the fabric warming container;

FIG. 3 is a diagrammatic view of a fabric warming container, according to various example embodiments;

FIG. 4 is a diagrammatic view of the fabric warming container of FIG. 3 with broken lines to show the interior elements of the fabric warming container;

FIG. 5 is a diagrammatic view of a fabric warming container, according to various example embodiments;

FIG. 6 is a diagrammatic view of the fabric warming container of FIG. 5 with broken lines to show the interior elements of the fabric warming container;

FIG. 7 is a diagrammatic view of a fabric warming container, according to various example embodiments;

FIG. 8 is a diagrammatic view of the fabric warming container of FIG. 7 with broken lines to show the interior elements of the fabric warming container; and

FIGS. 9-11 are diagrammatic views of a fabric warming container with a pair of sidewall portions coupled to and extending vertically from a base portion on opposing sides of the base portion.

Like reference symbols in the various drawings indicate like elements.

### DETAILED DESCRIPTION OF EXAMPLE EMBODIMENTS

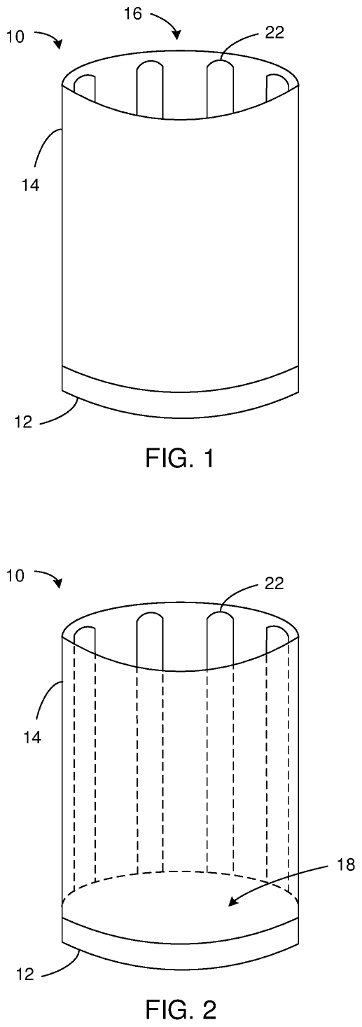

In general, consistent with the present disclosure, a fabric warming container is provided. For example, and referring generally to FIGS. 1-11, in some embodiments, a fabric warming container may include a base portion, one or more sidewall portions coupled to and extending vertically from the base portion along at least a portion of the periphery of the base portion. The one or more sidewall portions may include an interior surface. One or more ultraviolet (UV) light sources may be positioned on the interior surface of the one or more sidewall portions. As will be discussed in greater detail below, embodiments of the present disclosure may allow for fabrics, such as towels, to be warmed and sanitized without being completely enclosed.

Referring to FIGS. 1-11 and in some implementations, a fabric warming container (e.g., fabric warming container 10) may include a base portion (e.g., base portion 12). A container may generally include a receptacle configured to receive an article. A base portion (e.g., base portion 12) may generally include a generally solid structure configured to support the fabric warming container (e.g., fabric warming container 10). In some implementations, the base portion (e.g., base portion 12) may include weights to steady the fabric warming container (e.g., fabric warming container 10) from tipping over. In some implementations, the base portion may be formed from various materials (e.g., woods, plastics, fibers, metals, alloys, etc.) configured to support one or more sidewall portions and one or more ultraviolet (UV) light sources that may be positioned on an interior surface of the one or more sidewall portions. In some implementations, base portion 12 may include one or more legs configured to support fabric warming container 10. In some implementations and as will be discussed in greater detail below, base portion 12 may be built into a fixture (e.g., a wall, a door, etc.) and/or may be mountable to a fixture (e.g., a wall, a door, etc.).

In some implementations, the fabric warming container may include one or more sidewall portions coupled to and extending vertically from the base portion along at least a portion of the periphery of the base portion. For example, one or more sidewall portions (e.g., sidewall portion 14) may be coupled to the base portion (e.g., base portion 12) along at least a portion of the periphery of the base portion. In some implementations, the one or more sidewall portions (e.g., sidewall portion 14) may extend generally vertically (e.g., perpendicular to a horizontal plane formed by base portion 12) from base portion 12. In this manner, the one or more sidewall portions (e.g., sidewall portion 14) may form an opening (e.g., opening 16) opposite to the base portion (e.g., base portion 12) above a cavity (e.g., cavity 18) defined by the one or more sidewall portions (e.g., sidewall portion 14) and the base portion (e.g., base portion 12). In some implementations, the one or more sidewall portions may include an exterior surface and an interior surface. For example and in some implementations, the exterior surface of the one or more sidewall portions may include the surface of the sidewall portion(s) facing away from the base portion while the interior surface of the one or more sidewall portions may include the surface of the sidewall portion(s) facing towards the base portion. While sidewall portion 14 may extend substantially vertically from base portion 12, it will be appreciated that sidewall portion 14 may extend in any direction from the at least a portion of the periphery of base portion 12.

In some implementations, the base portion and sidewall portions may be integrally formed and/or may define a unitary structure. For example, base portion 12 and sidewall portion 14 may be integrally formed as a single structure. However and in some implementations, base portion 12 and sidewall portion 14 may be formed of distinct materials or combination of materials. For example, base portion 12 may be formed from a first material or first group of materials while sidewall portion 14 may be formed from a second material or second group of materials. In one example, fabric warming container may be a fabric warming “basket” formed with a base portion configured to weigh the fabric warming container against the ground while the sidewall portion(s) may be formed from a weaving of materials in a “basket” structure. However, it will be appreciated that base portion 12 and sidewall portion 14 may be formed from any combination of materials in various configurations within the scope of the present disclosure.

In some implementations, the cavity defined by the one or more sidewall portions and the base portion may be configured to receive one or more fabrics. For example and as shown in at least FIGS. 1-2, fabric warming container 10 may define a cavity (e.g., cavity 18) configured for receiving, holding, warming, and sanitizing fabrics. While fabric warming container 10 has been described for warming and/or sanitizing fabrics, it will be appreciated that any article may be received within the cavity (e.g., cavity 18) defined by the one or more sidewall portions (e.g., sidewall portion 14) and the base portion (base portion 12).

In some implementations, the one or more sidewall portions may include a single, continuous sidewall portion extending completely around the periphery of the base portion. Referring again to the examples of FIGS. 1-2 and in some implementations, fabric warming container 10 may include a single, continuous sidewall portion (e.g., sidewall portion 14) extending completely around the periphery of the base portion (e.g., base portion 12). In this manner, sidewall portion 14 may define an opening (e.g., opening 16) on an end of fabric warming container 10, opposite to base portion 12.

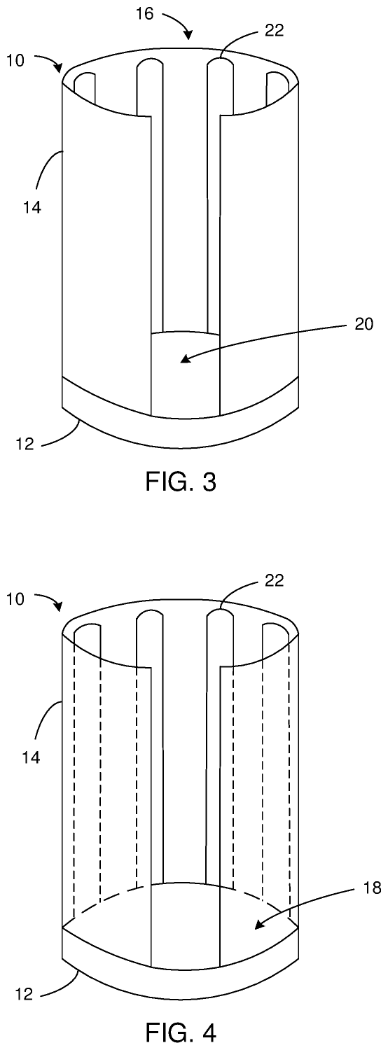

In some implementations, the one or more sidewall portions may include one or more gaps in the one or more sidewall portions extending from the opening along the sidewall portion to the base portion. Referring also to the examples of FIGS. 3-4 and in some implementations, sidewall portion 14 may include one or more gaps (e.g., gap 20) or space extending from the opening (e.g., opening 16) along sidewall portion 14 to base portion 12. In this manner, fabrics or other articles placed within fabric warming container 10 may be accessible through opening 16 and via gap 20. Accordingly, the contents of fabric warming container 10 may be accessible without having to reach from opening 16 all the way along or down the length of sidewall portion 14. While an example of one gap (e.g., gap 20) has been described that extends from opening 16 along sidewall portion 14 to base portion 12, it will be appreciated that the sidewall portion may include any number of holes or notches in various shapes that extend from any point of the sidewall portion to any other point of the sidewall portion within the scope of the present disclosure.

In some implementations, the one or more sidewall portions may include a plurality of sidewall portions spaced apart from one another along at least a portion of the periphery of the base portion. For example, while the above examples have included a single sidewall portion, it will be appreciated that fabric warming container 10 may include a plurality of sidewall portions spaced apart from one another (e.g., a varying distances, equivalent distances, etc.) within the scope of the present disclosure.

In some implementations, the fabric warming container may include one or more ultraviolet (UV) light sources positioned on the interior surface of the one or more sidewall portions. As is known in the art, UV light is a form of electromagnetic radiation that can sterilize exposed areas. Referring again to the example of FIGS. 1-2 and in some implementations, a UV light source (e.g., UV light source 22) may include any number of UV light sources positioned on the interior surface of the one or more sidewall portions. In this example, UV light source 22 may be positioned on the interior surface of sidewall portion 14 and configured to emit UV light within cavity 18. In this manner and as will be discussed in greater detail below, UV light source 22 may warm and sterilize fabric placed within fabric warming container 10. In some implementations, UV light source 22 may be built into sidewall portion 14 such that a light-emitting surface or portion of UV light source 22 may form a substantially unbroken surface with the interior surface of sidewall portion 14. In some implementations, UV light source 22 may be a directed UV light source (i.e., configured to emit UV light in a certain direction) and/or an undirected UV light source (i.e., configured to emit or diffuse UV light in every direction). As will be discussed in greater detail below, fabric warming container 10 may include a timer component configured to control the one or more UV light sources. While the examples of FIGS. 1-2 include e.g., four UV light sources extending vertically along sidewall portion 14, it will be appreciated that any number of UV light sources may be positioned on the interior surface of sidewall portion 14 and/or that the UV light sources may be configured in any orientation (e.g., vertically, horizontally, diagonally, etc.) within the scope of the present disclosure.

In some implementations, UV light source 22 may include an elongated, tubular or cylindrical UV light assembly extending along and/or within sidewall portion 14. However, it will be appreciated that UV light source 22 may include discrete UV light sources positioned along and/or within sidewall portion 14. It will also be appreciated that the discrete UV light sources may be configured to operate independently of one another. For example, a plurality of light emitting diode (LED) strips or segments may be used as UV light sources within the scope of the present disclosure and that each LED strip may be used independently of any other LED strip.

In some implementations, UV light source 22 may include one or more internal reflector portions or assemblies configured to reflect UV light in particular directions and/or away from UV light source 22. For example, fabric warming container 10 may include a plurality of UV light sources (e.g., UV light sources 22) positioned along sidewall portion 14 and configured to emit UV light within the cavity defined by the base portion and the one or more sidewall portions. In this example, UV light emitted by each of the UV light sources may be received by another UV light source. Accordingly, UV light source 22 may include one or more internal reflector portions or assemblies configured to reflect UV light received by an emitting UV light source away from the receiving UV light source. In this manner, UV light emitted and received by a plurality of UV light sources may be reflected away from UV light source 22 and back toward a fabric or other article placed within fabric warming container 10.

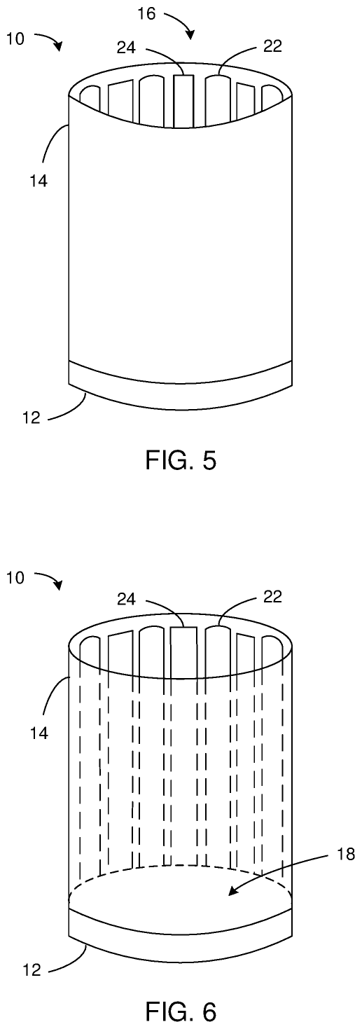

In some implementations, the fabric warming container may include one or more reflector portions positioned on the interior surface of the one or more sidewall portions. Referring also to the examples of FIGS. 5-6 and in some implementations, fabric warming container 10 may include one or more reflector portions (e.g., reflector portion 24) positioned on the interior surface of sidewall portion 14. As discussed above and in some implementations, the one or more UV light sources (e.g., UV light source 22) may emit UV light in multiple directions which may result in UV light being absorbed by portions of the interior surface of the one or more sidewall portions (e.g., sidewall portion 14) and not directed to fabrics or articles placed within the fabric warming container. Accordingly, fabric warming container 10 may include one or more reflector portions (e.g., reflector portion 24) positioned on the interior surface of sidewall portion 14 that are configured to reflect UV light back towards the center of fabric warming container 10. In some implementations, reflector portion 24 may be formed of a material configured to reflect UV light and may be shaped to reflect UV light away from reflection portion 24 (e.g., toward the center of fabric warming container 10). In this manner, reflector portion 24 may increase the UV light emission efficiency for fabrics or other articles placed within fabric warming container 10. While the examples of FIGS. 5-6 include e.g., three reflector portions extending vertically along the interior surface of sidewall portion 14, it will be appreciated that any number of reflector portions may be positioned on the interior surface of the one or more sidewall portions in any configuration (e.g., vertically, horizontally, diagonally, etc.) within the scope of the present disclosure.

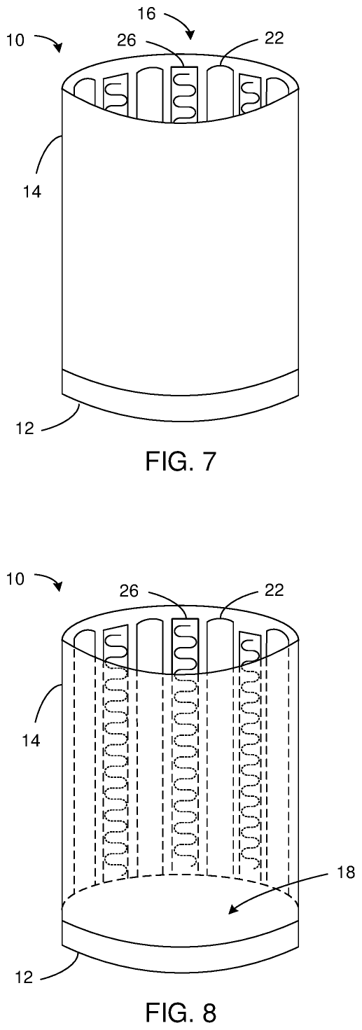

In some implementations, the fabric warming container may include one or more heating elements positioned on the interior surface of the one or more sidewall portions. Referring also to the examples of FIGS. 7-8 and in some implementations, in addition to the one or more UV light sources, fabric warming container 10 may include one or more heating elements (e.g., heating element 26) positioned on the interior surface of sidewall portion 14. In some implementations, the one or more heating elements (e.g., heating element 26) may be resistive heating elements. As is known in the art, a resistive heating element is configured to generate heat using electrical resistance. In this manner, the one or more resistive heating elements positioned on the interior surface of sidewall portion 14 may warm a fabric or other article placed within fabric warming container 10. In some implementations, heating element 26 may include one or more infrared light sources. As is known in the art, an infrared light source or infrared lamp emits infrared radiation. In this manner, infrared light sources may warm or heat fabric placed within fabric warming container 10. While examples of resistive heating elements and infrared light sources has been provided, it will be appreciated that various combinations of heating elements may be used within the scope of the present disclosure.

While the examples of FIGS. 7-8 include e.g., three heating elements extending vertically between corresponding UV light sources, it will be appreciated that any number of heating elements may be positioned on the interior surface of the one or more sidewall portions in any configuration (e.g., vertically, horizontally, diagonally, etc.) within the scope of the present disclosure. As will be discussed in greater detail below, fabric warming container 10 may include a timer component configured to control the one or more heating elements.

In some implementations, the fabric warming container may include a timer component configured to control the one or more UV light sources. For example, fabric warming container 10 may provide safety controls for the warming and/or sanitizing of fabrics. In some implementations, the timer component may include a default maximum amount of “on” time. For example, the timer component may be configured to activate the one or more UV light sources and may be configured to automatically shut off the one or more UV light sources after a threshold amount of time. The threshold amount of time may be user-defined or a default, pre-programmed value.

In some implementations, the timer component may be configured to activate the one or more heating elements and/or may be configured to automatically shut off the one or more heating elements after a threshold amount of time. In some implementations, the threshold amount of time may be different for the one or more heating elements versus the one or more UV light sources.

In some implementations, a power source for fabric warming container 10 may be provided by an external connection or an integral power source within fabric warming container 10. For example and in some implementations, a power source for the one or more UV light sources, one or more heating elements, etc. may include a power cable coupled to the base portion. In some implementations, fabric warming container 10 may be configured to be electrically coupled to a power source by hardwiring (e.g., a fixed electrical connection to a stationary power source), a removable plug, and/or connection to a battery or other rechargeable power supply. In some implementations, fabric warming container 10 may include an external power switch configured to switch power on and off to the one or more UV light sources, one or more heating elements, etc. In some implementations, fabric warming container 10 may include a first power switch for the one or more UV light sources and a second power switch for the one or more heating elements. While an example with two power switches has been provided, it will be appreciated that any number of power switches may be used within the scope of the present disclosure.

For example, fabric warming container 10 may include one or more power switches or controls to activate one or more heating elements to warm a fabric and one or more power switches or controls to activate one or more UV light sources to sanitize the fabric. In this manner, fabric warming container 10 may be configured (e.g., by operation of the one or more power switches or controls) to warm and/or sanitize a fabric or other article placed within fabric warming container 10. In some implementations, the one or more power switches or controls may activate the one or more heating elements and the one or more UV light sources together.

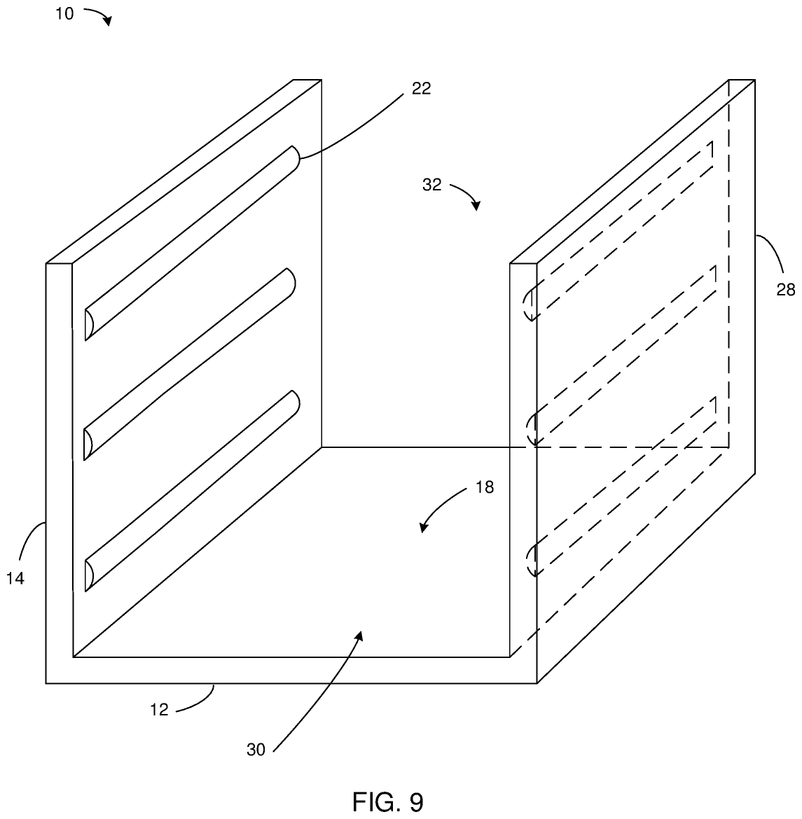

In some implementations, the fabric warming container may include a pair of sidewall portions coupled to and extending vertically from the base portion on opposing sides of the base portion. Referring also to the example of FIG. 9 and in some implementations, fabric warming container 10 may include a pair of sidewall portions (e.g., sidewall portions 14, 28) coupled to and extending vertically (e.g., perpendicular to the horizontal plane formed by base portion 12) from base portion 12 on opposing sides of base portion 12. In some implementations, the pair of sidewall portions (e.g., sidewall portions 14, 28) may form a pair of openings (e.g., openings 30, 32) positioned on opposing sides of base portion 12. For example and as shown in FIGS. 9-11, cavity 18 of fabric warming container 10 may be accessible from both opening 30 and opening 32. Accordingly, fabrics or other articles positioned within fabric warming container 10 may be accessible via opening 30 and/or opening 32.

In some implementations, the fabric warming container may include one or more ultraviolet (UV) light sources positioned on the interior surface of each sidewall portion. Referring again to the example of FIG. 9 and as discussed above, one or more UV light sources (e.g., UV light source 22) may be positioned on the interior surface of each sidewall portion (e.g., sidewall portions 14, 28). In this manner, the one or more UV light sources (e.g., UV light source 22) may be configured to emit UV light on and sanitize a fabric or other article placed in fabric warming container 10.

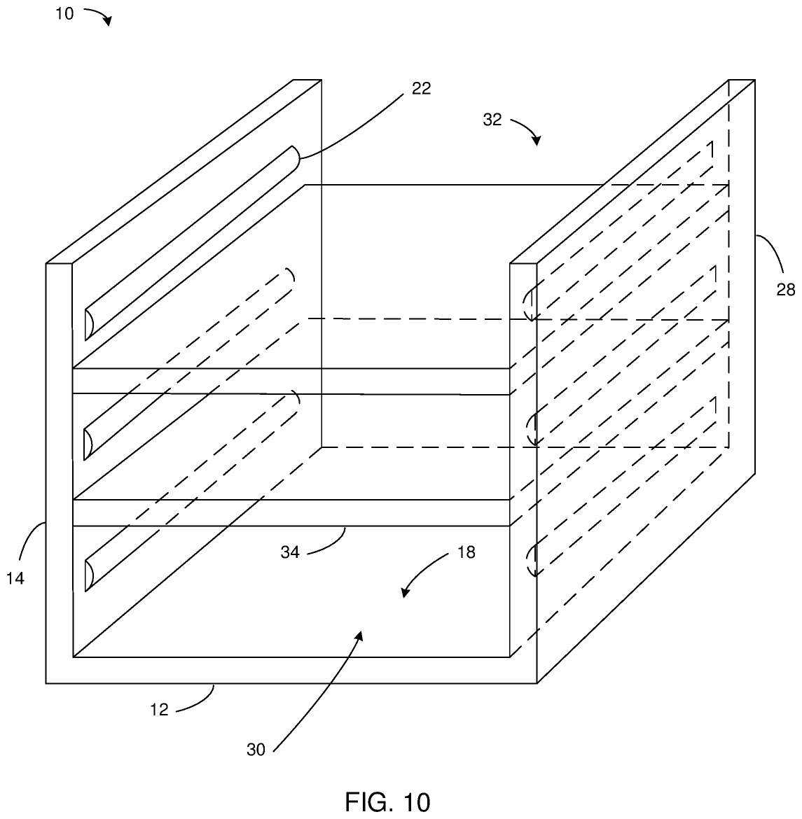

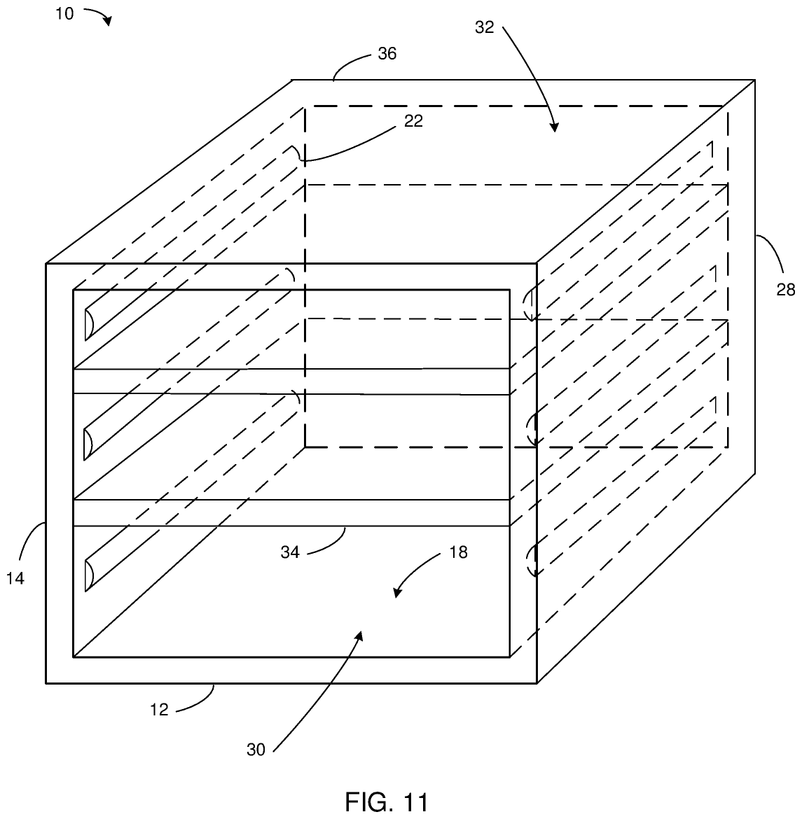

In some implementations, the fabric warming container may include one or more shelves positioned within the cavity of fabric warming container. Referring also to the example of FIGS. 10-11 and in some implementations, fabric warming container 10 may include one or more shelves (e.g., shelf 34) positioned within the cavity (e.g., cavity 18) defined by sidewall portions 14 and 28. In this manner, fabrics or other articles may be organized on the one or more shelves (e.g., shelf 34) to more efficiently utilize cavity 18 for sanitizing as many fabrics and/or other articles as can fit within fabric warming container 10. While the example of FIGS. 10-11 show e.g., two shelves extending horizontally between sidewall portions 14 and 28, it will be appreciated that any number of shelves may be positioned within fabric warming container 10 and may be configured in various configurations (e.g., horizontal shelves, vertical shelves, diagonal shelves, etc.). It will also be appreciated that other structures or racking may be used to position fabrics and/or other articles within fabric warming container 10 within the scope of the present disclosure. For example and as shown in the example of FIG. 11, fabrics or other articles may be draped on or otherwise allowed to hang from a structure or structures extending from a top portion (e.g., top portion 36) extending over the pair of sidewall portions.

In some implementations, the fabric warming container may be configured to be one or more of: coupled to an article of furniture, built into a wall, and mounted onto a wall. For example and in some implementations, fabric warming container 10 may be configured to be coupled to an article of furniture (e.g., a dresser, a chair, a couch, a desk, as part of a shelving unit, or any other type or article of furniture or fixture). In this manner, fabric warming container 10 may be coupled to or built-into an article of furniture or other fixture to provide access to warmed and sanitized fabrics or other articles.

In some implementations, the fabric warming container may be configured to be built into a wall. Referring again to the examples of FIGS. 9-11 and in some implementations, fabric warming container 10 may be configured to be built into a fixture (e.g., a wall, a door, etc.). In some implementations, fabric warming container 10 may define a built-in container or receptacle configured to receive warm and/or sanitize fabrics and/or other articles placed therein. In one example, fabric warming container 10 may be built into a wall and accessible from at least one opening. In another example, fabric warming container 10 may be built into a wall and accessible from two openings (e.g., openings on opposite sides of the wall as shown in the example of FIG. 11 where cavity 18 is accessible via opening 30 on one side and opening 32 on the opposite side.

In some implementations, the fabric warming container may be configured to be mounted onto a wall. Referring again to the example of FIGS. 9-11, fabric warming container 10 may be mounted onto a wall in the form of one or more “shelves” configured to warm and/or sanitize fabrics and/or other articles placed within the fabric warming container. While an example of mounting to a wall has been provided, it will be appreciated that fabric warming container 10 may be mounted to any fixture within the scope of the present disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A fabric warming container comprising:
a base portion;
one or more sidewall portions coupled to and extending vertically from the base portion along at least a portion of the periphery of the base portion, the one or more sidewall portions including an interior surface; and
one or more ultraviolet (UV) light sources positioned on the interior surface of the one or more sidewall portions,
wherein the one or more sidewall portions form an opening opposite to the base portion above a cavity defined by the one or more sidewall portions and the base portion.

2. The fabric warming container of claim 1, further comprising:
one or more heating elements positioned on the interior surface of the one or more sidewall portions.

3. The fabric warming container of claim 1, further comprising:
one or more reflector portions positioned on the interior surface of the one or more sidewall portions.

4. The fabric warming container of claim 1, wherein the cavity defined by the one or more sidewall portions and the base portion is configured to receive one or more fabrics.

5. The fabric warming container of claim 1, wherein the one or more sidewall portions include a single, continuous sidewall portion extending completely around the periphery of the base portion.

6. The fabric warming container of claim 1, wherein the one or more sidewall portions include a plurality of sidewall portions spaced apart from one another along at least a portion of the periphery of the base portion.

7. The fabric warming container of claim 1, wherein the one or more sidewall portions include one or more gaps in the one or more sidewall portions extending from the opening along the sidewall portion to the base portion.

8. The fabric warming container of claim 1, further comprising:
a timer component configured to control the one or more UV light sources.

9. The fabric warming container of claim 1, further comprising:
one or more shelves positioned within the cavity defined by the one or more sidewall portions.

10. The fabric warming container of claim 1, wherein the fabric warming container is configured to be one or more of: coupled to an article of furniture, built into a wall, and mounted onto a wall.

11. A fabric warming container comprising:
a base portion;
a pair of sidewall portions coupled to and extending vertically from the base portion on opposing sides of the base portion, each sidewall portion including an interior surface;
one or more ultraviolet (UV) light sources positioned on the interior surface of each sidewall portion,
wherein the pair of sidewall portions form a pair of openings positioned on opposing sides of the base portion.

12. The fabric warming container of claim 11, further comprising:
one or more heating elements positioned on the interior surface of at least one of the sidewall portions.

13. The fabric warming container of claim 11, further comprising:
one or more reflector portions positioned on the interior surface of at least one of the sidewall portions.

14. The fabric warming container of claim 11, further comprising:
a timer component configured to control the one or more UV light sources.

15. The fabric warming container of claim 11, further comprising:
one or more shelves positioned between the pair of sidewall portions.

16. The fabric warming container of claim 11, wherein the fabric warming container is configured to be one or more of: coupled to an article of furniture, built into a wall, and mounted onto a wall.

17. A fabric warming container comprising:
a base portion;
one or more sidewall portions coupled to and extending vertically from the base portion along at least a portion of the periphery of the base portion, the one or more sidewall portions including an interior surface; and
one or more ultraviolet (UV) light sources positioned on the interior surface of the one or more sidewall portions,
wherein the one or more sidewall portions and the base portion define a cavity within the fabric warming container, and
wherein the fabric warming container is configured to be one or more of: coupled to an article of furniture, built into a wall, and mounted onto a wall.

18. The fabric warming container of claim 17, further comprising:
one or more heating elements positioned on the interior surface of the one or more sidewall portions.

19. The fabric warming container of claim 17, further comprising:
one or more shelves positioned within the cavity defined by the one or more sidewall portions.

20. The fabric warming container of claim 17, further comprising:
one or more reflector portions positioned on the interior surface of at least one of the sidewall portions.
